(19) United States
(12) Patent Application Publication
(10) Pub. No.: US 2013/0012465 A1
(43) Pub. Date: Jan. 10, 2013

(54) BIBW 2992 FOR USE IN THE TREATMENT OF TRIPLE NEGATIVE BREAST CANCER

(75) Inventors: Christian Haslinger, Vienna (AT); Flavio Solca, Vienna (AT); Martina Uttenreuther-Fischer, Biberach an der Riss (DE)

(73) Assignee: Boehringer Ingelheim International GmbH, Ingelheim am Rhein (DE)

(21) Appl. No.: 13/511,172

(22) PCT Filed: Dec. 6, 2010
(86) PCT No.: PCT/EP2010/068968

§ 371 Date: Jun. 20, 2012

(30) Foreign Application Priority Data
Dec. 7, 2009 (EP) 09178179.9

(51) Int. Cl. A61K 31/517; A61K 31/664; A61P 35/00; A61K 31/704

(52) U.S. Cl. 514/34; 514/266.24; 514/110

(57) ABSTRACT

The present invention relates to a method of treating patients suffering from triple negative breast cancer comprising administration of an effective amount of the irreversible EGFR/HER1 and HER2 inhibitor BIBW 2992 (1) to a person in need of such treatment, optionally in combination with the administration of a further chemotherapeutic agent (2), in combination with radiotherapy, radio-immunotherapy and/or tumour resection by surgery.

The present invention relates to a method of treating patients suffering from triple negative breast cancer comprising administration of an effective amount of the irreversible EGFR/HER1 and HER2 inhibitor BIBW 2992 (1) to a person in need of such treatment, optionally in combination with the administration of a further chemotherapeutic agent (2), in combination with radiotherapy, radio-immunotherapy and/or tumour resection by surgery.

### BACKGROUND OF THE INVENTION

BIBW 2992 is known as the compound 4-[(3-chloro-4-fluorophenyl)amino]-6-{[4-(N,N-dimethylamino)-1-oxo-2-buten-1-yl]amino}-7-((S)-tetrahydro furan-3-yloxy)-quinazo line,

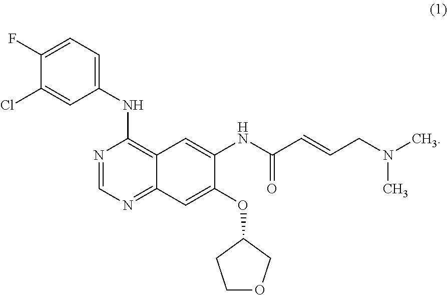 [SMILES: CN(C)C/C=C/C(=O)Nc1cc2c(Nc3ccc(F)c(Cl)c3)ncnc2cc1O[C@H]1CCOC1]

BIBW 2992 is a potent and selective dual inhibitor of erbb1 receptor (EGFR) and erbB2 (Her2/neu) receptor tyrosine kinases. Furthermore, BIBW 2992 was designed to covalently bind to EGFR and HER2 thereby irreversibly inactivating the receptor molecule it has bound to. This compound, salts thereof such as the dimaleate salt, their preparation as well as pharmaceutical formulations comprising BIBW 2992 or a salt thereof, indications to be treated with BIBW 2992 and combinations including BIBW 2992 are disclosed in WO 02/50043, WO 2005/037824, WO 2007/054550 and WO 2007/054551.

Breast cancers are classified by whether or not they express respectively overexpress the genes for the estrogen receptor, progesterone receptor or HER2/neu (ERBB2). These three receptors are known to promote proliferation of breast cancer cells, and the most successful breast cancer treatments are hormone-based or antibody-based drugs that directly target these receptors. It is important to know what subtype the cancer is before commencing treatment as different drugs target different receptors. Triple-negative breast cancer (TNBC) refers to a specific subtype of breast cancer that does not express sufficient protein amounts of the genes coding for estrogen receptor (ER), progesterone receptor (PR) and HER2/neu. This subtype of breast cancer is clinically characterised as more aggressive and less responsive to standard (receptor-mediated) treatment, including Herceptin and Tamoxifen, and associated with poorer overall patient prognosis. Depending upon the stage of diagnosis, triple negative breast cancer can be extremely aggressive, and more likely to metastasize than other subtypes of breast cancer. Histologically, such cancers are poorly differentiated, and most fall into the basal subgroup of breast cancers, characterised by staining for basal markers (ie, cytokeratin 5/6). It is diagnosed more frequently in younger (premenopausal) women, women with BRCA1 mutations, and in African-American and Hispanic ethnic groups. Triple-negative breast cancer accounts for approximately 15% of all breast cancer cases.

After surgery and radiation treatments, TNBC is typically responsive to chemotherapy—a combination of three medications called ACT, which stands for Adriamycin, cyclophosphamide (Cytoxan), and taxanes (e.g. paclitaxel, docetaxel).

### SUMMARY OF THE INVENTION

Surprisingly, the irreversible EGFR/HER1 and HER2 inhibitor BIBW2992 (1) is advantageously effective in the treatment of patients suffering from triple negative breast cancer.

Thus, as a first aspect the present invention relates to a method of treating patients suffering from triple negative breast cancer comprising administering an effective amount of the irreversible EGFR/HER1 and HER2 inhibitor BIBW 2992 (1) to a person in need of such treatment, optionally in combination with the administration of a further chemotherapeutic agent L21, and/or optionally in combination with radiotherapy, radio-immunotherapy and/or tumour resection by surgery.

A second aspect of the present invention is BIBW 2992 (1), or a salt thereof, for the treatment of a patient suffering from triple negative breast cancer, e.g. a pharmaceutical composition comprising BIBW 2992 (1) for the treatment of a patient suffering from triple negative breast cancer.

A third aspect of the present invention is the use of BIBW 2992 (1) for preparing a pharmaceutical composition for the treatment of a patient suffering from triple negative breast cancer.

In a first preferred embodiment of the present invention the method of treatment according to the invention is neoadjuvant/adjuvant treatment of triple negative breast cancer.

In a second preferred embodiment of the present invention the indication to be treated is triple negative metastatic breast cancer.

In a third preferred embodiment of the present invention the method of treatment according to the invention is a 1^{st} line treatment after failure of neoadjuvant or adjuvant chemotherapy or without prior exposure to neoadjuvant/adjuvant chemotherapy in case of primary metastatic disease.

In a fourth preferred embodiment of the present invention the method of treatment according to the invention is a 2nd line treatment after failure of one prior chemotherapy.

In a fifth preferred embodiment of the present invention the method of treatment according to the invention is a 3rd line treatment after failure of two different prior chemotherapies.

In a sixth preferred embodiment of the present invention the method of treatment according to the invention is a combination treatment comprising administering an effective amount of BIBW 2992 (1), in combination with the administration of an effective amount of one, two or three of the chemotherapeutics selected from Adriamycin, cyclophosphamide (Cytoxan), taxanes (e.g. paclitaxel, docetaxel) and platinum compounds (e.g. cisplatin, carboplatin, oxaliplatin, satraplatin, tetraplatin or iproplatin), preferably after surgery and radiation treatments, to a person in need of such treatment.

In a seventh preferred embodiment of the present invention the method of treatment according to the invention is a single agent treatment with BIBW 2992 (1), or a combination treatment comprising administering an effective amount of BIBW 2992 (1) in combination with an effective amount of an established or experimental chemotherapeutic agent such as a further chemotherapeutic agent (2) or one, two or three of the chemotherapeutics selected from adriamycin, cyclophosphamide (cytoxan), taxanes (e.g. paclitaxel, docetaxel) and platinum compounds (e.g. cisplatin, carboplatin, oxaliplatin, satraplatin, tetraplatin or iproplatin), to a specific and preselected triple negative breast cancer patient with demonstrated tumoral erbB receptor dysfunction. These particular patients harbor tumors with erbB receptor (HER1, HER3 and/or HER4) or cognate ligand (EGF TGFa, AREG, Hb-EGF, BTC, Epigen, EREG, NRG1, NRG2, NRG3, NRG4, Tomoregulin and neurglycan) overexpression or mutation that can be detected at the protein, mRNA or DNA level using methods familiar to people skilled to the art. Alternatively, tumoral erbB receptor dysfunction can be be detected by hallmarks of erbB receptor activation (e.g phosphorylated EGFR, phosphorylated HER2, phosphorylated HER3, phosphorylated HER4, homo- or heterodimerized EGFR, homo- or heterodimerized HER2, heterodimerized HER3, homo- or heterodimerized HER4) using methods familiar to people skilled to the art.

Response to the treatment or clinical benefit (complete response [CR], partial response [PR], or stable disease [SD]) is monitored by methods well known to those of skill in the art, applying response evaluation criteria in solid tumours (RECIST) criteria, as described by P. Therasse et al., J Natl Cancer Inst 2000, 92, 205-216, and by Eisenhauer E A, Therasse P, Bogaerts J, Schwartz L H, Sargent D, Ford R, et al., New response evaluation criteria in solid tumours: revised RECIST guideline (version 1.1). Eur J Cancer 2009; 45:228-247. For example, the results of treatment, i.e. objective response or tumour shrinkage, stable disease or progression of the tumour diesease, may be monitored by way of visual inspection of the cancer, such as, by means of clinical assessment, X-ray, CT scan or MRI. Alternatively, progression may be monitored by way of tumor biomarker detection.

In one embodiment, the patient is monitored at various time points throughout the treatment of the cancer. For example, the progression of a cancer may be monitored by analyzing the progression of cancer at a second time point and comparing this analysis to an analysis at a first time point. The first time point may be before or after initiation of BIBW 2992 (1) treatment and the second time point is after the first. An increased growth of the cancer indicates progression of the cancer.

### DETAILED DESCRIPTION OF THE INVENTION

The method of treatment according to the invention comprises administration of a therapeutically effective amount of BIBW 2992 (1) or a pharmaceutically acceptable salt thereof, optionally in combination with the administration of a further chemotherapeutic agent (2), to a patient in need thereof, optionally in combination with radiotherapy, radio-immunotherapy and/or tumour resection by surgery. Dosages or amounts of actives provided in the context of this invention refer in any case to the free base equivalent, that is BIBW 2992 (1) in the free base form.

Within the context of the invention, (1) is optionally applied in the form of the tautomers and pharmaceutically acceptable salts thereof. Pharmaceutically acceptable salts are preferably selected from the group consisting of the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrolactate, hydroxalate, hydrosuccinate, hydrobenzoate and hydro-p-toluenesulphonate, preferably the hydrochloride, hydrobromide, hydrosulphate, hydrophosphate, hydromaleate, hydrofumarate and hydromethanesulphonate. In a particularily preferred embodiment (1) is applied as its hydromaleate, preferably in the ratio (1): maleic acid=1:2 as depicted in formula (1a) below.

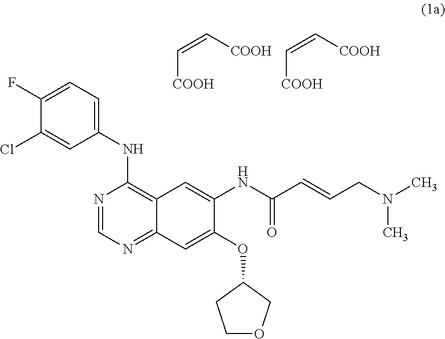 [SMILES: CN(C)C/C=C/C(=O)Nc1cc2c(Nc3ccc(F)c(Cl)c3)ncnc2cc1O[C@H]1CCOC1 ; O=C(O)/C=C\C(=O)O ; O=C(O)/C=C\C(=O)O]

Within the meaning of the present invention, the following classes of chemotherapeutic agents (2) are especially of interest, although not representing a limitation:
Synthetic small molecule VEGF receptor antagonists
Small molecule growth factor (GF) receptor antagonists including FGFR, HGFR or PDGFR
Small molecule inhibitors of down stream signaling kinases such as c-src family members or FAK
Inhibitors of the EGF receptor and/or HER2 receptors and/or VEGF receptor and/or integrin receptors or any other protein tyrosine kinase receptors, which are not classified under the synthetic small-molecules
Small molecule Polo-like kinase-1 (PLK-1) inhibitors
Small molecule inhibitors of the Ras/Raf/MAPK pathway including MEK inhibitors
Small molecule inhibitors of the PI3K/AKT pathway including mTOR inhibitors or any other serine/threonine kinases.
Inhibitors of the Ras/Raf/MAPK or PI3K/AKT pathways or any other serine/threonine kinases, which are not classified under the synthetic small-molecules
Inhibitors directed to EGF receptor and/or VEGF receptor and/or integrin receptors or any other protein tyrosine kinase receptors, which are synthetically manufactured antibodies, antibody fragments or fusion proteins
Inhibitors directed to circulating VEGF, which are synthetically manufactured antibodies, antibody fragments or fusion proteins
Inhibitors directed to the IGF1 receptor and/or IGF1 or IGF2 growth factor, which are synthetically manufactured chemical entities or antibodies, antibody fragments or fusion proteins
Compounds which interact with nucleic acids and which are classified as alkylating agents or platinum compounds
Compounds which interact with nucleic acids and which are classified as anthracyclines, as DNA intercalators or as DNA cross-linking agents
Anti-metabolites
Inhibitors of DNA repair enzymes including for instance PARP inhibitors
Naturally occurring, semi-synthetic or synthetic bleomycin type antibiotics (BLM-group antibiotics)
Inhibitors of DNA transcribing enzymes, especially topoisomerase I or topoisomerase II inhibitors
Chromatin modifying agents
Mitosis inhibitors, anti-mitotic agents, or cell-cycle inhibitors
Compounds interacting with or binding tubulin
Compounds inhibiting mitotic kinesins or other motor proteins including but not limited to Eg5, CENP-E, MCAK, Kid, MKLP-1
Proteasome inhibitors
Heat shock protein inhibitors
Compounds targeting the anti-apoptotic function of Bcl-2, Bcl-x₁ and like molecules
Enzymes Hormones, hormone antagonists or hormone inhibitors, or inhibitors of steroid biosynthesis
Steroids
Cytokines, hypoxia-selective cytotoxins, inhibitors of cytokines, lymphokines, antibodies directed against cytokines or oral and parenteral tolerance induction strategies
Supportive agents
Antiinflammatory compounds such as but not limited to COX-2 inhibitors
Chemical radiation sensitizers and protectors
Photochemically activated drugs
Synthetic poly- or oligonucleotides
Other chemotherapeutic or naturally occurring, semi-synthetic or synthetic therapeutic agents, such as cytotoxic antibiotics, antibodies targeting surface molecules of cancer cells, antibodies targeting growth factors or their receptors, inhibitors of metalloproteinases, inhibitors of oncogenes, inhibitors of gene transcription or of RNA translation or protein expression, or complexes of rare earth elements.

In another preferred embodiment of the invention the chemotherapeutic agent (2) is selected from the group consisting of a small molecule VEGF receptor antagonist such as vatalanib (PTK-787/ZK222584), 3-Z-[1-(4-(N-((4-methyl-piperazin-1-yl)-methylcarbonyl)-N-methyl-amino)-anilino)-1-phenyl-methylene]-6-methoxycarbonyl-2-indolinone (BIBF 1120) or a salt thereof, preferably BIBF 1120 monoethanesulphonate, SU-5416, SU-6668, SU-11248, SU-14813, AZD-6474, AZD-2171, CP-547632, CEP-7055, AG-013736, IM-842 or GW-786034, small molecule EGFR and HER2 antagonist such as PF-299,804, AZD-8931, AC-480, BMS-690514, AV-412, HKI-272, CI-1033 or GW-2016, an EGFR antagonist such as iressa (gefitinib, ZD-1839), tarceva (erlotinib, OSI-774), PKI-166, or EKB-569, an antagonist of the mitogen-activated protein kinase such as BAY-43-9006 or BAY-57-9006, a protein kinase receptor antagonist which is not classified under the synthetic small molecules such as atrasentan, rituximab, trastuzumab (herceptin), pertuzumab (2C4), cetuximab (erbitux, C-225), zalutumumab (HuMax EGFr), panitumumab (Vectibix), nimotuzumab (h-R3) and matuzumab (EMD-72000), MM-111, MM-121, mAb806, mAb175, AMG888/U3-1287, MM-12, Avastin™ (bevacizumab), bivatuzumab mertansine, IMC-1C11, DC-101, vitaxin, imatinib, a protein tyrosine kinase inhibitor which is a fusion protein such as VEGFtrap, an alkylating agent or a platinum compound such as melphalan, cyclophosphamide (Cytoxan), an oxazaphosphorine, cisplatin, carboplatin, oxaliplatin, satraplatin, tetraplatin, iproplatin, mitomycin, streptozocin, carmustine (BCNU), lomustine (CCNU), busulfan, ifosfamide, streptozocin, thiotepa, chlorambucil, a nitrogen mustard such as mechlorethamine, an ethyleneimine compound, an alkylsulphonate, daunorubicin, doxorubicin (adriamycin), liposomal doxorubicin (doxil), epirubicin, idarubicin, mitoxantrone, amsacrine, dactinomycin, distamycin or a derivative thereof, netropsin, pibenzimol, mitomycin, CC-1065, a duocarmycin, mithramycin, chromomycin, olivomycin, a phtalanilide such as propamidine or stilbamidine, an anthramycin, an aziridine, a nitrosourea or a derivative thereof, a pyrimidine or purine analogue or antagonist or an inhibitor of the nucleoside diphosphate reductase such as cytarabine, 5-fluorouracile (5-FU), pemetrexed, tegafur/uracil, uracil mustard, fludarabine, gemcitabine, capecitabine, mercaptopurine, cladribine, thioguanine, methotrexate, pentostatin, hydroxyurea, or folic acid, a phleomycin, a bleomycin or a derivative or salt thereof, CHPP, BZPP, MTPP, BAPP, liblomycin, an acridine or a derivative thereof, a rifamycin, an actinomycin, adramycin, a camptothecin such as irinotecan (camptosar) or topotecan, an amsacrine or analogue thereof, a tricyclic carboxamide, an histonedeacetylase inhibitor such as SAHA, MD-275, trichostatin A, CBHA, LAQ824, or valproic acid, an anti-cancer drug from plants such as paclitaxel (taxol), docetaxel or taxotere, a vinca alkaloid such as navelbine, vinblastin, vincristin, vindesine or vinorelbine, a tropolone alkaloid such as colchicine or a derivative thereof, a macrolide such as maytansine, an ansamitocin or rhizoxin, an antimitotic peptide such as phomopsin or dolastatin, an epipodophyllotoxin or a derivative of podophyllotoxin such as etoposide or teniposide, a steganacin, an antimitotic carbamate derivative such as combretastatin or amphetinile, procarbazine, a proteasome inhibitor such as bortezomib, an enzyme such as asparaginase, pegylated asparaginase (pegaspargase) or a thymidine-phosphorylase inhibitor, a gestagen or an estrogen such as estramustine (T-66) or megestrol, an anti-androgen such as flutamide, casodex, anandron or cyproterone acetate, an aromatase inhibitor such as aminogluthetimide, anastrozole, formestan or letrozole, a GNRH analogue such as leuprorelin, buserelin, goserelin or triptorelin, an anti-estrogen such as tamoxifen or its citrate salt, droloxifene, trioxifene, raloxifene or zindoxifene, a derivative of 17β-estradiol such as ICI 164,384 or ICI 182,780, aminoglutethimide, formestane, fadrozole, finasteride, ketoconazole, a LH-RH antagonist such as leuprolide, a steroid such as prednisone, prednisolone, methylprednisolone, dexamethasone, budenoside, fluocortolone or triamcinolone, an interferon such as interferon β, an interleukin such as IL-10 or IL-12, an anti-TNFα antibody such as etanercept, TNFα(tasonermin) an immunomodulatory drug such as thalidomide, its R- and S-enantiomers and its derivatives, or revimid (CC-5013), a leukotrien antagonist, mitomycin C, an aziridoquinone such as BMY-42355, AZQ or EO-9, a 2-nitroimidazole such as misonidazole, NLP-1 or NLA-1, a nitroacridine, a nitroquinoline, a nitropyrazoloacridine, a “dual-function” nitro aromatic such as RSU-1069 or RB-6145, CB-1954, a N-oxide of nitrogen mustard such as nitromin, a metal complex of a nitrogen mustard, an anti-CD3 or anti-CD25 antibody, a tolerance induction agent, a biphosphonate or derivative thereof such as minodronic acid or its derivatives (YM-529, Ono-5920, YH-529), zoledronic acid monohydrate, ibandronate sodium hydrate or clodronate disodium, a nitroimidazole such as metronidazole, misonidazole, benznidazole or nimorazole, a nitroaryl compound such as RSU-1069, a nitroxyl or N-oxide such as SR-4233, an halogenated pyrimidine analogue such as bromodeoxyuridine, iododeoxyuridine, a thiophosphate such as WR-2721, a photo-chemically activated drug such as porfimer, photofrin, a benzoporphyrin derivative, a pheophorbide derivative, merocyanin 540 (MC-540) or tin etioporpurin, an ant-template or an anti-sense RNA or DNA such as oblimersen, a non-steroidal inflammatory drug such as acetylsalicyclic acid, mesalazin, ibuprofen, naproxen, flurbiprofen, fenoprofen, fenbufen, ketoprofen, indoprofen, pirprofen, carprofen, oxaprozin, pranopro-fen, miroprofen, tioxaprofen, suprofen, alminoprofen, tiaprofenic acid, fluprofen, indomethacin, sulindac, tolmetin, zomepirac, nabumetone, diclofenac, fenclofenac, alclofenac, bromfenac, ibufenac, aceclofenac, acemetacin, fentiazac, clidanac, etodolac, oxpinac, mefenamic acid, meclofenamic acid, flufenamic acid, nifluminic acid, tolfenamic acid, diflunisal, flufenisal, piroxicam, tenoxicam, lornoxicam, nimesulide, meloxicam, celecoxib, rofecoxib, or a pharmaceutically acceptable salt of a non-steroidal inflammatory drug, a cytotoxic antibiotic, an antibody targeting the surface molecules of cancer cells such as apolizumab or 1D09C3, an inhibitor of metalloproteinases such as TIMP-1 or TIMP-2, Zinc, an inhibitor of oncogenes such as P53 and Rb, a complex of rare earth elements such as the heterocyclic complexes of lanthanides, a photo-chemotherapeutic agent such as PUVA, an inhibitor of the transcription factor complex ESX/DRIP130/Sur-2, an inhibitor of HER-2 expression, such as the heat shock protein HSP90 modulator geldanamycin and its derivative 17-allylaminogeldanamycin or 17-AAG, or a therapeutic agent selected from cytoxan, IM-842, tetrathiomolybdate, squalamine, combrestatin A4, TNP-470, marimastat, neovastat, bicalutamide, abarelix, oregovomab, mitumomab, TLK-286, alemtuzumab, ibritumomab, temozolomide, denileukin diftitox, aldesleukin, dacarbazine, floxuridine, plicamycin, mitotane, pipobroman, plicamycin, tamoxifen and testolactone, furthermore inhibitors of DNA repair enzymes including for instance PARP inhibitors selected from BSI-201, veliparib, PF-01367338, olaparib, INO-1001.

In accordance with the present invention BIBW 2992 (1) and the optional chemotherapeutic may be administered by oral (including buccal or sublingual), enterical, parenteral (e.g., intramuscular, intraperitoneal, intravenous, transdermal or subcutaneous injection, or implant), nasal, vaginal, rectal, or topical (e.g. inhalative) routes of administration and may be formulated, alone or together, in suitable dosage unit formulations containing conventional non-toxic pharmaceutically acceptable carriers, adjuvants and vehicles appropriate for each route of administration.

In a preferred embodiment BIBW 2992 (1) is administered orally, enterically, transdermally, intravenously, peritoneally or by injection, preferably orally. Dosage forms and formulations suitable within the present invention are known in the art. For instance, such dosage forms and formulations include those disclosed for BIBW 2992 (1) in WO 02/50043, WO 2007/054550 and WO 2007/054551 or in the Examples of the subject application.

### Dosages/BIBW 2992:

In one embodiment the invention relates to the method of treatment described above, characterised in that BIBW 2992 (1), or its polymorph, metabolite, hydrate, solvate, or a pharmaceutically acceptable salt thereof, is administered intermittent or in a daily dosage such that the plasma level of the active substance preferably lies between 10 and 5000 nM for at least 12 hours of the dosing interval.

For oral treatment BIBW 2992 (1) may be administered to the human patient in a daily dose of 0.01-4 mg/kg of body weight (bw), preferably 0.1-2 mg/kg, particularly preferred in a dose of 0.2-1.3 mg/kg bw. For instance, BIBW 2992 (1) may be administered orally in a total daily dose of 10 to 150 mg, preferably 20 to 70 mg, most preferred 40 to 60 mg. For example, the daily oral dosis administered may be 10, 20, 30, 40, 50, 60, 70, 100 or 150 mg, most preferred is a total daily dose of 50 mg. The daily dosage may optionally be divided into multiple doses, e.g. 1, 2 or 3 doses to be administered through the day. Preferably the oral daily dose is administered only once a time.

The dosage for intravenous use of BIBW 2992 (1) may be 1-500 mg, preferably 5-300 mg, particularly preferred 10-100 mg, either given as a bolus or, especially if higher doses are applied, as a slow intravenous infusion over several hours, e.g. over about 1, 2, 4, 6, 10, 12 or 24 hours.

### Dosages/Chemotherapeutic Agents (2):

Pharmaceutical compositions, dosages and treatment schedules for the individual chemotherapeutic agents (2) are known in the art and may be applied analogously within the invention. Depending on the individual activity of the specific combination dosage of the chemotherapeutic agents (2) may be reduced, e.g. may vary in the range of 1/1 to 1/20 of the dosages described in the prior art.

For patients with metastatic breast cancer the combination with docetaxel may be given at a dose between 55 mg/m² and 100 mg/m² and most specifically at a dose of 60 to 75 mg/m² in administration schedule of once every 21 days. In a weekly administration schedule the dose of docetaxel may be lowered.

For patients with metastatic breast cancer the combination with adriamycin (doxorubicin) may be given at a dose between 50 mg/m² and 80 mg/m² and most specifically at a dose of 60 to 75 mg/m² in administration schedule of once every 21 days. In a weekly administration schedule the dose of doxorubicin may be lowered.

For patients with metastatic breast cancer the combination with cyclophosphamide (cytoxan) may be given intravenously as 40 to 50 milligrams (mg) per 2.2 pounds of body weight in divided doses over a period of 2 to 5 days. It may also be given as 10 to 15 mg per 2.2 pounds of body weight every 7 to 10 days or 3 to 5 mg per 2.2 pounds of body weight twice weekly.

In patients with metastatic breast cancer, the administration of paclitaxel (taxol) is at a dose of up to 175 mg/m² over 3 hours every 3 weeks. In a weekly administration schedule paclitaxel dose may be lower, e.g 80 mg/m². In an adjuvant setting, paclitaxel will be administered at doses up to 175 mg/m² over 3 hours every 3 weeks sequentially to a combination with a doxorubicin-containing chemotherapy (four courses of doxorubicin and cyclophosphamide were used).

In patients with breast cancer, vinorelbine may be used at a dose of up to 25 mg/m² in a weekly administration schedule.

In patients with metastatic breast cancer, gemcitabine at a dose of 1250 mg/m² over 30 minutes on days 1 and 8 of each 21-day treatment cycle will be used in combination with paclitaxel. Paclitaxel should be administered at 175 mg/m² as a 3-hour infusion before the administration of gemcitabine on day 1.

However, it may optionally be necessary to deviate from the amounts specified for BIBW 2992 (1) and/or the individual chemotherapeutic agents (2), depending on the body weight or method of administration, the individual response to the medication, the nature of the formulation used and the time or interval over which it is administered. Thus, in some cases, it may be sufficient to use less than the minimum quantity specified above, while in other cases the upper limit specified will have to be exceeded. When large amounts are administered it may be advisable to spread them over the day in a number of single doses.

### Dosages/Radiotherapy or Radio-Immunotherapy:

Dosages and treatment schedules for radiotherapy and radio-immunotherapy are known in the art and may be applied analogously within the invention. Depending on the individual activity of the specific combination with BIBW 2992 (1) and, optionally, chemotherapeutic agent (2) dosage of the radiotherapy and radio-immunotherapy component may be reduced, e.g. may vary in the range of 1/1 to 1/20 of the dosages described in the art.

### DEFINITIONS

The terms “ErbB 1”, “epidermal growth factor receptor” and “EGFR” are used interchangeably herein and refer to native sequence EGFR as disclosed, for example, in Carpenter et al. Ann Rev. Biochem. 56:881-914 (1987), including variants thereof (e.g. a deletion mutant EGFR as in Humphrey et al. PNAS (USA) 87:4207-4211 (1990)). erbB1 refers to the gene encoding the EGFR protein product. As used herein, the EGFR protein is disclosed as GenBank accession no. NP_{—}005219 which is encoded by the erbB1 gene, GenBank accession no. NM_{—}005228. The sequences are disclosed as SEQ ID NO: 1, and SEQ ID NO: 2, respectively, in FIG. 5 of WO 2006/084058.

The term “therapeutically effective amount” shall mean that amount of a drug or pharmaceutical agent that will elicit the biological or medical response of a tissue system, animal or human that is being sought by a researcher or clinician, resulting in a beneficial effect for at least a statistically significant fraction of patients, such as a improvement of symptoms, a cure, a reduction in disease load, reduction in tumor mass or cell numbers, stable disease, extension of life, or improvement in quality of life.

The following Examples serve to illustrate the invention without restricting it:

### Example 1

### Clinical Study

### Methods

### Study Design

A Phase II, open-label, multicenter trial was conducted in Belgium and Germany. Two Cohorts of patients were included:
Cohort A: Patients with triple negative mBC
Cohort B: Patients with HER2-negative, HR-positive mBC.
Primary endpoint for Cohort A and B initially was objective response as determined by response evaluation criteria in solid tumours (RECIST) criteria, as described by P. Therasse et al., J Natl Cancer Inst 2000, 92, 205-216. For Cohort A this was modified by amendment to clinical benefit (CB), i.e. complete response (CR), partial response (PR) and stable disease (SD) for at least 4 courses.
Secondary endpoints were time to progression, progression-free survival (PFS), overall survival (OS), time to objective response, duration of objective response, safety and pharmacokintics (PK). Clinical benefit (CR, PR, SD) was a secondary endpoint for Cohort B.
A total of 80 patients was planned to be enrolled into the trial: 80, i.e. 40 patients per cohort. An early stopping rule was implemented after 20 patients in each cohort.
Response to treatment was assessed at 8 weeks and at 8-week intervals thereafter.
For quantification of BIBW 2992 plasma concentrations 5 ml of venous blood were collected on Day 1 of Course 1 and Day 15 of Course 2 at the following time points: predose and 1, 2 and 3 hours after drug administration. In addition, a voluntary PK sample could be requested within 4 to 24 hours after BIBW 2992 administration. Additional predose plasma samples were taken on Day 15 of Course 1, on Day 1 of Course 2 and on Day 1 of all subsequent courses (Course 3 onwards). BIBW 2992 drug concentrations were determined by a validated high performance liquid chromatography-mass spectroscopy (HPLC-MS/MS) assay. The distribution of BIBW 2992 plasma concentrations was summarised by timepoint by descriptive statistics, and if feasible, was graphically inspected.

### Treatment

Patients received a single daily oral dose of BIBW 2992 (50 mg/day) until disease progression or withdrawal due to adverse events (AEs). Dose reduction to 40 mg/day and 30 mg/day was an option for patients experiencing certain drug-related AEs.

### Main Eligibility Criteria

Confirmed diagnosis of Stage IV HER2-negative metastatic breast cancer (HER22+ and fluorescence in situ hybridization-negative or HER21+ or negative)
Progression following no more than two lines of chemotherapy including adjuvant, not including hormone treatments for Cohort B
Age ≧18 years
Life expectancy of at least 6 months
Written informed consent
Eastern Cooperative Oncology Group (ECOG) performance score 0-2
Measurable disease according to RECIST criteria
Availability of tumour samples (fresh or paraffin material) for EGFR-testing.
No prior treatment with EGFR/HER1 or HER2 inhibiting agent
Adequate liver, kidney and bone marrow function
No concomitant treatment with other investigational drugs
No other anti-cancer-therapy within four weeks prior to the first treatment with the trial drug.
Concurrent treatment with biphosphonates is allowed.

### Additional Inclusion Criteria for Cohort A

HR-negative patients: estrogen receptor (ER) status and progesterone receptor (PgR) status must be assessed by immunohistochemistry (IHC) and Allred (Harvey J M et al., J Clin Oncol 1999, 17(5), 1474-1481).

### Additional Inclusion Criteria for Cohort B

ER-positive and/or PgR-positive patients: ER-status and PgR-status must be assessed by IHC and Allred (Harvey J M et al., J Clin Oncol 1999, 17(5), 1474-1481).

### Results

From 1 Dec. 2006 to 20 May 2009, a total of 56 patients were screened for this study.
A total of 50 patients have been treated with BIBW 2992 on the study. 29 patients were included into cohort A (triple negative) and 21 patients were included into cohort B (HER2-negative, HR-positive). All of the patients included into Cohort B were ER-positive, 19 of 21 were also PgR-positive.
Nineteen patients discontinued treatment due to AEs (15 due to dose-reducing toxicities and four due to other AEs). Thirty-one patients discontinued due to disease progression (19 patients in Cohort A and 12 patients in Cohort B).
Patients were on treatment for a mean of 59 days (range: 6-336 days). Time on treatment was slightly longer in Cohort A, with a mean of 62 days versus 54 days in Cohort B.

### Response Assessment

27 patients in Cohort A and 18 patients in Cohort B were assessed at least once for response to treatment.
No objective responses were observed in either cohort.

### Cohort A:

No objective response (OR) was observed, but durable clinical benefit (CB) was seen in two patients early in the study and, following an amendment, this cohort was allowed to recruit to full accrual.
Seven patients had a best response of SD.
A total of three patients remained on treatment with SD for >4 cycles, with SD maintained for 132 to 335 days

### Cohort B:

No OR was observed. According to an early stopping rule recruitment was terminated on 1 Feb. 2008.
5 patients had a best response of SD.
1 patient had SD for ≧4 cycles.
   PFS and survival data for cohorts A and B are depicted in FIG. 1.

### SUMMARY AND CONCLUSIONS

A total of 50 patients have been treated in this Phase II study
BIBW 2992 has a safety profile similar to other EGFR TKIs. The most frequently observed drug-related side-effects are gastrointestinal and skin related AEs, which were mostly manageable.
Pharmacokinetics appear to be similar to the data observed in previous Phase I trials.
Among 21 patients included in Cohort B (hormone receptor positive), no OR was observed and following an early stopping rule, recruitment was terminated early.
With regard to Cohort A, as durable SD has been observed in two patients early on, the trial was amended to allow for full accrual if at least 3/20 patients showed clinical benefit. A total of 29 patients with triple negative breast cancer were included into cohort A. Overall median PFS was 53 days. However, for the three patients, who had clinical benefit for >4 months, mean PFS was 217 days, with a median at 184 days.
BIBW 2992 showed antitumour activity in some patients with advanced triple-negative breast cancer.

### Example 2

### Preclinical data

TABLE 1
Breast cancer: triple negative breast cell panel

| | BIBW 2992 | | lapatinib | |
|---|---|---|---|---|
| | EC₅₀ | EC₅₀ | EC₅₀ | EC₅₀ |
| Model | [nM] 2D | [nM] 3D | [nM] 2D | [nM] 3D |
| SUM 149PT | — | 17 | — | 1356 |
| HCC 1806 | >4000 | 47 | >4000 | >4000 |
| HCC 1143 | >4000 | >1000 | >4000 | >4000 |
| BT20 | >4000 | 23 | >4000 | 467 |
| HCC 1937 | >4000 | >4000 | >4000 | >4000 |
| MDA-MB-231 | >10000 | 2154 | >10000 | >4000 |
| CAMA-1 | 234 | 277 | >1000 | 3010 |
| HCC 70 | 571 | >1000 | >1000 | >1000 |
| MDA-MB-468 | — | 1249 | — | >1000 |
| BT 549 | 2024 | 956 | >1000 | — |

TABLE 2
Breast cancer/triple negative, EGFR positive SUM149 Xenografts in mice

| | | T/C [%] |
|---|---|---|
| Compound | | Day 42 |
| | Dose* | |
| | [mg/kg/d] | |
| BIBW 2992 | 20 | 5 |
| BIBW 2992 | 20 | 9 |
| erlotinib | 75 | 6 |
| lapatinib | 2 × 100 | 25 |
| HKI-272 | 50 | 36 |

*corresponds to MTD for these compounds

| | | |
|---|---|---|
| | Dose | |
| | [mg/kg/q2w] | |
| Herceptin | 10 | 153 |

SUM 149 express large amounts of EGFR, AREG, EREG and TGFα

### Example 3

### Pharmaceutical Compositions of Solid BIBW 2992 MA₂ Tablets

(MA₂: dimaleinate)

TABLE 1

| | Formulation | | | | |
|---|---|---|---|---|---|
| | A | B | C | D | E |
| | mg per | mg per | mg per | mg per | mg per |
| Ingredient | tablet | tablet | tablet | tablet | tablet |
| BIBW 2992 MA₂, unmilled | 29.5600 | 44.3400 | 59.1200 | 73.9000 | 103.4600 |
| (= BIBW 2992 base) | (20.0000) | (30.0000) | (40.0000) | (50.0000) | (70.0000) |
| Lactose monohydrate | 123.8600 | 185.7900 | 247.7200 | 309.6500 | 433.5100 |
| Microcrystalline cellulose | 18.4800 | 27.7200 | 36.9600 | 46.2000 | 64.6800 |
| Crospovidone | 3.6000 | 5.4000 | 7.2000 | 9.0000 | 12.6000 |
| Colloidal anhydrous silica | 0.9000 | 1.3500 | 1.8000 | 2.2500 | 3.1500 |
| Magnesium stearate | 3.6000 | 5.4000 | 7.2000 | 9.0000 | 12.6000 |
| Total | 180.0000 | 270.0000 | 360.0000 | 450.0000 | 630.0000 |

Formulations A, B and C, D and E are tablets which can be coated with a film-coat according to Table 2.

TABLE 2
Exemplary composition of filmcoatings for formulation A-E

| | Coating for Formulation | | | | |
|---|---|---|---|---|---|
| | A | B | C | D | E |
| Ingredient | mg per tablet | | | | |
| Hypromellose | 2.5000 | 3.5000 | 4.0000 | 5.0000 | 6.0000 |
| Polyethylene glycol 400 | 0.5000 | 0.7000 | 0.8000 | 1.0000 | 1.2000 |
| Titanium dioxid | 1.1300 | 0.6825 | 1.8080 | 0.9750 | 1.1700 |
| Indigo Carmine | 0.0700 | 0.2450 | 0.1120 | 0.3500 | 0.4200 |
| aluminum lacquer | | | | | |
| Talcum | 0.6500 | 1.6625 | 1.0400 | 2.3750 | 2.8500 |
| Polysorbate 80 | 0.1500 | 0.2100 | 0.2400 | 0.3000 | 0.3600 |
| Purified water | — | — | — | — | — |
| (volatile component) | | | | | |
| Total | 5.0000 | 7.0000 | 8.0000 | 10.0000 | 12.0000 |

### LEGEND TO THE FIGURES

FIG. 1:

Kaplan-Meier curve of overall survival by cohort A and B treated set, referring to Example 1.

A: progression free survival

B: overall survival.

FIG. 2:

Tumour volume [mm³] of breast cancer/triple negative, EGFR positive SUM149 Xenografts in mice upon treatment with 75 mg/kg erlotinib, against control (vehicle)

FIG. 3:

Tumour volume [mm³] of breast cancer/triple negative, EGFR positive SUM149 Xenografts in mice upon treatment with 20 mg/kg BIBW 2992 BS (dosage provided for the free base), in comparison to 2×200 mg/kg lapatinib, against control (vehicle)

FIG. 4:

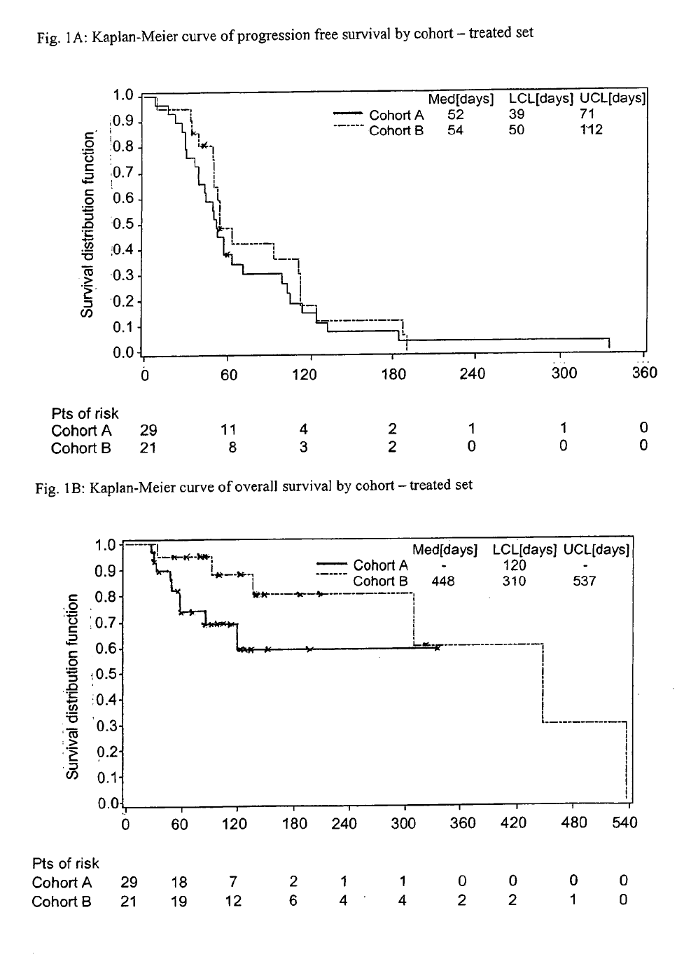

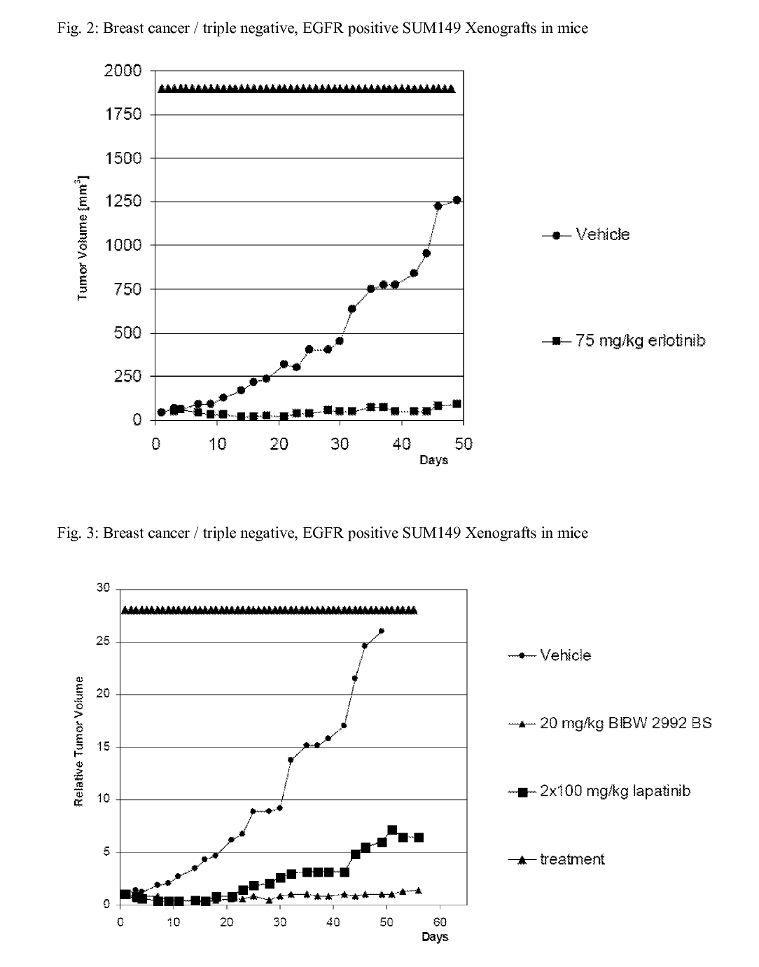

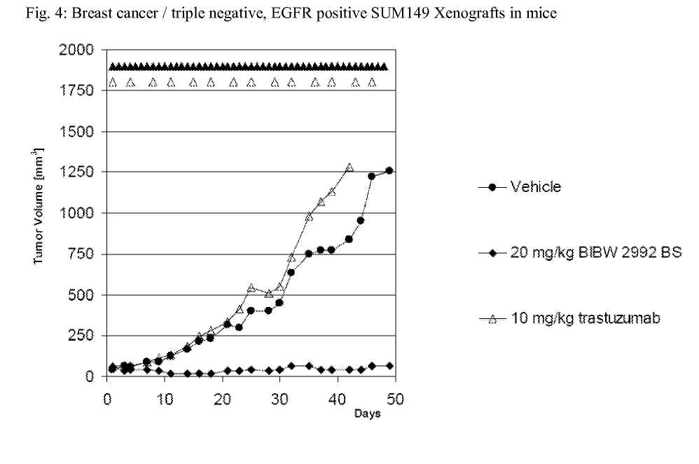

Tumour volume [mm³] of breast cancer/triple negative, EGFR positive SUM149 Xenografts in mice upon treatment with 20 mg/kg BIBW 2992 BS (dosage provided for the free base), in comparison to 10 mg/kg trastuzumab, against control (vehicle)

1. A method of treating patients suffering from triple negative breast cancer comprising administering an effective amount of the irreversible EGFR inhibitor BIBW 2992 (1), or a salt thereof, to a person in need of such treatment, optionally in combination with the administration of a further chemotherapeutic agent (2), and/or optionally in combination with radiotherapy, radio-immunotherapy and/or tumour resection by surgery.

2. The method of claim 1 wherein the treatment is neoadjuvant or adjuvant treatment.

3. The method of claim 1 wherein the cancer is triple negative metastatic breast cancer.

4. The method of claim 1, wherein the treatment is a 1^{st} line treatment after failure of neoadjuvant or adjuvant chemotherapy or without prior exposure to neoadjuvant/adjuvant chemotherapy in case of primary triple negative metastatic breast cancer or
the treatment is a 2^{nd} line treatment after failure of one prior chemotherapy or
the treatment is a 3^{rd} line treatment after failure of two different prior chemotherapies.

5. The method of claim 1 wherein the treatment is a combination treatment comprising administering BIBW 2992 (1), in combination with the administration of one, two or three of the chemotherapeutics selected from Adriamycin, cyclophosphamide (Cytoxan), taxanes and platinum compounds after surgery and radiation treatments, to a person in need of such treatment.

6. The method of claim 1 wherein the treatment is a combination treatment comprising administering BIBW 2992 (1) in combination with a further chemotherapeutic agent (2) selected from
Synthetic small molecule VEGF receptor antagonists,
Small molecule growth factor (GF) receptor antagonists FGFR, HGFR or PDGFR,
Small molecule inhibitors of down stream signaling kinases selected from c-src family members and FAK,
Inhibitors of the EGF receptor and/or HER2 receptors and/or VEGF receptor and/or integrin receptors or any other protein tyrosine kinase receptors, which are not classified under the synthetic small-molecules,
Small molecule Polo-like kinase-1 (PLK-1) inhibitors,
Small molecule inhibitors of the Ras/Raf/MAPK pathway,
Small molecule inhibitors of the PI3K/AKT pathway mTOR inhibitors or any other serine/threonine kinases,
Inhibitors of the Ras/Raf/MAPK or PI3K/AKT pathways or any other serine/threonine kinases, which are not classified under the synthetic small-molecules,
Inhibitors directed to EGF receptor and/or VEGF receptor and/or integrin receptors or any other protein tyrosine kinase receptors, which are synthetically manufactured antibodies, antibody fragments or fusion proteins,
Inhibitors directed to circulating VEGF, which are synthetically manufactured antibodies, antibody fragments or fusion proteins,
Inhibitors directed to the IGF1 receptor and/or IGF1 or IGF2 growth factor, which are synthetically manufactured chemical entities or antibodies, antibody fragments or fusion proteins,
Compounds which interact with nucleic acids and which are classified as alkylating agents or platinum compounds,
Compounds which interact with nucleic acids and which are classified as anthracyclines, as DNA intercalators or as DNA cross-linking agents,
Anti-metabolites,
Inhibitors of DNA repair enzymes,
Naturally occurring, semi-synthetic or synthetic bleomycin type antibiotics (BLM-group antibiotics),
Inhibitors of DNA transcribing enzymes, especially topoisomerase I or topoisomerase II inhibitors,
Chromatin modifying agents,
Mitosis inhibitors, anti-mitotic agents, or cell-cycle inhibitors,
Compounds interacting with or binding tubulin,
Compounds inhibiting mitotic kinesins or other motor proteins selected from Eg5, CENP-E, MCAK, Kid; and MKLP-1,
Proteasome inhibitors,
Heat shock protein inhibitors,
Compounds targeting the anti-apoptotic function of Bcl-2, Bcl-x₁,
Enzymes Hormones, hormone antagonists or hormone inhibitors, or inhibitors of steroid biosynthesis,
Steroids,
Cytokines, hypoxia-selective cytotoxins, inhibitors of cytokines, lymphokines, antibodies directed against cytokines or oral and parenteral tolerance induction strategies,
Supportive agents,
Antiinflammatory compounds,
Chemical radiation sensitizers and protectors,
Photochemically activated drugs,
Synthetic poly- or oligonucleotides, and
cytotoxic antibiotics, antibodies targeting surface molecules of cancer cells, antibodies targeting growth factors or their receptors, inhibitors of metalloproteinases, inhibitors of oncogenes, inhibitors of gene transcription or of RNA translation or protein expression, or complexes of rare earth elements,
or in combination with one, two or three of the chemotherapeutics selected from adriamycin, cyclophosphamide (cytoxan), taxanes and platinum compounds to a preselected patient suffering from triple negative breast cancer with demonstrated tumoral erbB receptor dysfunction.

7. The method of claim 6 wherein the tumoral erbB receptor dysfunction is characterized by erbB receptor (HER1, HER3 and/or HER4) or cognate ligand (EGF TGFa, AREG, Hb-EGF, BTC, Epigen, EREG, NRG1, NRG2, NRG3, NRG4, Tomoregulin and neurglycan) overexpression or mutation that can be detected at the protein, mRNA or DNA level.

8-21. (canceled)
